(19) United States
(12) Patent Application Publication
(10) Pub. No.: US 2007/0049839 A1
(43) Pub. Date: Mar. 1, 2007

(54) SYSTEM AND METHOD FOR AUTOMATED AIRWAY EVALUATION FOR MULTI-SLICE COMPUTED TOMOGRAPHY (MSCT) IMAGE DATA USING AIRWAY LUMEN DIAMETER, AIRWAY WALL THICKNESS AND BRONCHO-ARTERIAL RATIO

(75) Inventors: Benjamin Odry, West New York, NJ (US); Atilla Peter Kiraly, Plainsboro, NJ (US); Carol L. Novak, Newton, PA (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS CORPORATE RESEARCH INC., PRINCETON, NJ (US)

(21) Appl. No.: 11/465,548

(22) Filed: Aug. 18, 2006

Related U.S. Application Data
(60) Provisional application No. 60/713,024, filed on Aug. 31, 2005.

(51) Int. Cl. A61B 5/08

(52) U.S. Cl. 600/529

(57) ABSTRACT

A method for evaluating an airway in a bronchial tree, includes: segmenting a bronchial tree; modeling the segmented bronchial tree; computing a first ratio for an airway in the segmented and modeled bronchial tree, wherein the first ratio is a ratio between a diameter of the airway lumen and a diameter of an artery accompanying the airway; computing a second ratio for the airway, wherein the second ratio is a ratio between the diameter of the artery and a thickness of the airway wall; or computing a tapering index for the airway, wherein the tapering index indicates a tapering of the diameter of the airway lumen; scoring and color coding the first ratio, second ratio or tapering index; and visualizing the segmented and modeled bronchial tree color coded according to the first ratio, second ratio or tapering index.

### CROSS-REFERENCE TO RELATED APPLICATION

This application claims the benefit of U.S. Provisional Application No. 60/713,024, filed Aug. 31, 2005, a copy of which is herein incorporated by reference.

### BACKGROUND OF THE INVENTION

1. Technical Field

The present invention relates to medical image processing, and more particularly, to a system and method for automated airway evaluation for multi-slice computed tomography (MSCT) image data using airway lumen diameter, airway wall thickness and broncho-arterial ratio.

2. Discussion of the Related Art

Pulmonary diseases such as bronchiectasis, asthma and emphysema are characterized by abnormalities in airway dimensions. Multi-slice computed tomography (MSCT) has become one of the primary means to depict these abnormalities as the availability of high-resolution near-isotropic data makes it possible to evaluate airways at angles that are oblique to a scanning plane. However, clinical evaluation of the airways is generally limited to visual inspection. Such systematic evaluation of the airways has proved to be impractical without automation.

Computerized measurements of airway lumen diameter and airway wall thickness are critical for airway disease management. The usefulness of a scoring system for the health status of the airways using thin section computed tomography has been proven as described in Bhalla M., Turcios N., Aponte V., Jenkins M., Leitman B., McCauley D. and Naidich D). (1991) “Cystic Fibrosis: Scoring system with Thin Section CT” Radiology 1991 179:783-788.

The use of multidetector computed-tomography (MDCT) was reported in Odry B. L., Kiraly A. P., Novak C. L., Naidich D. P., Lerallut J-F (2005) “A visualization tool for global evaluation of bronchiectasis and local evaluation of the airways” European Medical & biological Engineering conference EMBEC'05 Proceedings; Prague, November 2005, as being promising for the evaluation of bronchiectasis.

Various bronchial tree segmentation methods have been proposed. One method described in Kiraly A. P., McLennan G., Hoffman E. A., Reinhardt J. M., and Higgins W. E., (2002) “Three-dimensional human airway segmentation methods for clinical virtual bronchoscopy” Academic Radiology, 2002. 9(10): p. 1153-1168 and Kiraly A. P., Helferty J. P., Hoffman E. A., McLennan G. and Higgins W. E. (2004) “Three dimensional path planning for virtual bronchoscopy” in IEEE Transactions on Medical Imaging. vol. 23, no. 1, November 2004: p. 1365-1379, uses an adaptive region growing based segmentation starting from a seed point.

A morphology-based method described in Fetita C. I., Preteux F., Beigelman-Aubry C., and Grenier P., (2004) “Pulmonary airways: 3-D reconstruction from multislice CT and clinical investigation” vol. 23, no. 11, IEEE Trans. Medical Imaging, November 2004 and Aykac D, Hoffman E A, McLennan G, and Reinhardt J M, (2003) “Segmentation and analysis of the human airway tree from three-dimensional X-ray CT images” IEEE Trans. Medical Imaging, 22(8):940-950, August 2003, can lead to detailed segmentations; however, its processing time can take up to an hour. In addition, its segmentation targets airways of all sizes by applying multiple scale operators to the entire lung region.

An alternative segmentation presented in Kiraly A. P., McLennan G., Hoffman E. A., Reinhardt J. M., and Higgins W. E., (2002) “Three-dimensional human airway segmentation methods for clinical virtual bronchoscopy” Academic Radiology, 2002. 9(10): p. 1153-1168, combines an initial adaptive region growing followed by a small-scale morphology to target small airways.

In a tracking-based approach presented in Tschirren J, Hoffman E A, McLennan G, and Sonka M, (2004) “Airway tree segmentation using adaptive regions of interest” Medical Imaging 2004: Physiology, Function, and Structure from Medical Images, Vol. 5369, an entire airway tree is tracked from the trachea to terminal branches with the segmentation adapting to several different scales present in these generations.

A level-set approach based on analyzing the front of a shape was proposed in Schlatholter, T., Lorenz, C., Carlsen, I., Renisch, S. and Deschamps, T. (2002) “Simultaneous segmentation and tree reconstruction of the airways for virtual bronchoscopy.” Image Processing. Volume 4684 of SPIE Medical Imaging. (2002) 103-113.

Systems described in Wiemker R., Blaffert T., Bulow T., Renisch S. and Lorenz C. (2004): “Automated assessment of bronchial lumen, wall thickness and bronchioarterial diameter ratio of the tracheobronchonchial tree using high-resolution CT” Computer Assisted Radiology and Surgery 2004, International Congress Series 1268 967-972, Berger P., Perot V., Desbarats P., Tunon-de-Lara J. M., Marthan R. and Laurent F. (2005) “Airway wall Thickness in Cigarette Smokers: Quantitative Thin-Section CT Assessment”, Radiology 2005 235:1055-1064, Kiraly A. P., Reinhardt J. M., Hoffman E. A., McLennan G and Higgins W. E., (2005) “Virtual bronchoscopy for quantitative airway analysis”, SPIE Conf. on Medical Imaging, vol. 5746, pg. 369-383, 2005 and Grenier P., Maurice F., Musset D., Menu Y. and Nahum H. (1986) “Bronchiectasis: assessment by thin section CT.” Radiology 1986 161:95-99, provide automated assessment of airway parameters such as airway lumen diameter, airway wall diameter and/or broncho-arterial ratio; but, besides plots, lack any associated visualization to localize abnormalities.

### SUMMARY OF THE INVENTION

In an exemplary embodiment of the present invention, a method for evaluating an airway in a bronchial tree, comprises: segmenting a bronchial tree; modeling the segmented bronchial tree; computing a first ratio for an airway in the segmented and modeled bronchial tree, wherein the first ratio is a ratio between a diameter of the airway lumen and a diameter of an artery accompanying the airway; computing a second ratio for the airway, wherein the second ratio is a ratio between the diameter of the artery and a thickness of the airway wall; or computing a tapering index for the airway, wherein the tapering index indicates a tapering of the diameter of the airway lumen; scoring and color coding the first ratio, second ratio or tapering index; and visualizing the segmented and modeled bronchial tree color coded according to the first ratio, second ratio or tapering index.

Segmenting the bronchial tree comprises applying a filtered adaptive threshold region growing to the bronchial tree starting from a seed point in a trachea. Modeling the segmented bronchial tree comprises: defining a skeleton of the segmented bronchial tree; performing a multistage refinement of the skeleton to arrive at a tree structure; and computing a diameter map of the tree structure.

The diameter of the airway lumen and the thickness of the airway wall are determined by: computing a centerline of the airway; computing a three-dimensional (3D) gradient of a volume of the airway within a first threshold; positioning a tube along the centerline; iteratively expanding the tube by increasing its radius until the radius of the tube reaches the first threshold; determining inner and outer radii of the tube by checking the 3D gradient computed along an x-axis and a y-axis of the tube at a boundary of the tube at each iteration; and fitting the tube to the airway by using the determined inner and outer radii, wherein the inner radius of the fit tube is half the diameter of the airway lumen and the outer radius of the fit tube minus the inner radius of the fit tube is the thickness of the airway wall.

The artery is identified and the diameter of the artery are determined by: labeling regions of high intensity in a cross-sectional plane of the bronchial tree; computing a score based on a circularity of the region, similarity with the airway and proximity to the airway, wherein a region with a highest score is the artery; and computing a mean distance from a center of the artery to boundary points of the artery, wherein the mean distance is half the diameter of the artery.

The tapering index is determined by: plotting the diameter of the airway lumen as a function of voxels along a path from a trachea to a terminal branch of the bronchial tree along which the airway is situated; and computing a slope the diameter of the airway lumen along the path, wherein the tapering index relates to the computed slope.

The first ratio, second ratio and tapering index are scored by: setting a score of the first ratio according to a value of the first ratio; setting a score of the second ratio according to a value of the second ratio; and setting a score of the tapering index according to a value of the tapering index. The method further comprises color-coding the first ratio, second ratio and tapering index according to the value of the first ratio, second ratio and tapering index, respectively. The method further comprises: acquiring an image of a chest including the bronchial tree by using computed tomography or magnetic resonance imaging.

In an exemplary embodiment of the present invention, a system for evaluating an airway in a bronchial tree, comprises: a memory device for storing a program; a processor in communication with the memory device, the processor operative with the program to: segment a bronchial tree; model the segmented bronchial tree; compute a first ratio for an airway in the segmented and modeled bronchial tree, wherein the first ratio is a ratio between a diameter of the airway lumen and a diameter of an artery accompanying the airway; compute a second ratio for the airway, wherein the second ratio is a ratio between the diameter of the artery and a thickness of the airway wall; or compute a tapering index for the airway, wherein the tapering index indicates a tapering of the diameter of the airway lumen; score and color code the first ratio, second ratio or tapering index; and visualize the segmented and modeled bronchial tree color coded according to the first ratio, second ratio or tapering index.

When segmenting the bronchial tree the processor is further operative with the program to apply a filtered adaptive threshold region growing to the bronchial tree starting from a seed point in a trachea. When modeling the segmented bronchial tree the processor is further operative with the program to: define a skeleton of the segmented bronchial tree; perform a multistage refinement of the skeleton to arrive at a tree structure; and compute a diameter map of the tree structure.

When determining the diameter of the airway lumen and the thickness of the airway wall the processor is further operative with the program to: compute a centerline of the airway; compute a 3D gradient of a volume of the airway within a first threshold; position a tube along the centerline; iteratively expand the tube by increasing its radius until the radius of the tube reaches the first threshold; determine inner and outer radii of the tube by checking the 3D gradient computed along an x-axis and a y-axis of the tube at a boundary of the tube at each iteration; and fit the tube to the airway by using the determined inner and outer radii, wherein the inner radius of the fit tube is half the diameter of the airway lumen and the outer radius of the fit tube minus the inner radius of the fit tube is the thickness of the airway wall.

When the artery is identified and the diameter of the artery are determined the processor is further operative with the program to: label regions of high intensity in a cross-sectional plane of the bronchial tree; compute a score based on a circularity of the region, similarity with the airway and proximity to the airway, wherein a region with a highest score is the artery; and compute a mean distance from a center of the artery to boundary points of the artery, wherein the mean distance is half the diameter of the artery.

When determining the tapering index the processor is further operative with the program to: plot the diameter of the airway lumen as a function of voxels along a path from a trachea to a terminal branch of the bronchial tree along which the airway is situated; and compute a slope the diameter of the airway lumen along the path, wherein the tapering index relates to the computed slope.

When scoring the first ratio, second ratio and tapering index the processor is further operative with the program to: set a score of the first ratio according to a value of the first ratio; set a score of the second ratio according to a value of the second ratio; and set a score of the tapering index according to a value of the tapering index.

The processor is further operative with the program code to color-code the first ratio, second ratio and tapering index according to the value of the first ratio, second ratio and tapering index, respectively. The processor is further operative with the program code to acquire an image of a chest including the bronchial tree by using a computed tomography or magnetic resonance imaging device.

In an exemplary embodiment of the present invention, a method for automatically evaluating multi-slice computed tomography (MSCT) image data of a bronchial tree, comprises: segmenting and modeling the bronchial tree starting from a trachea; computing a first ratio for each airway of the bronchial tree, wherein the first ratio is a ratio between a diameter of the airway lumen and a diameter of an artery accompanying the airway; scoring and color coding the first ratio; visualizing the segmented and modeled bronchial tree color coded according to the first ratio; computing a second ratio for each airway of the bronchial tree, wherein the second ratio is a ratio between the diameter of the artery and a thickness of the airway wall; scoring and color coding the second ratio; visualizing the segmented and modeled bronchial tree color coded according to the second ratio; computing a tapering index for each airway of the bronchial tree, wherein the tapering index indicates a tapering of the diameter of the airway lumen; scoring and color coding the tapering index; and visualizing the segmented and modeled bronchial tree color coded according to the tapering index.

The diameter of the airway lumen and the thickness of the airway wall are determined by: computing a centerline of the airway; computing a 3D gradient of a volume of the airway within a first threshold; positioning a tube along the centerline; iteratively expanding the tube by increasing its radius until the radius of the tube reaches the first threshold; determining inner and outer radii of the tube by checking the 3D gradient computed along an x-axis and a y-axis of the tube at a boundary of the tube at each iteration; and fitting the tube to the airway by using the determined inner and outer radii, wherein the inner radius of the fit tube is half the diameter of the airway lumen and the outer radius of the fit tube minus the inner radius of the fit tube is the thickness of the airway wall.

The artery is identified and the diameter of the artery are determined by: labeling regions of high intensity in a cross-sectional plane of the bronchial tree; computing a score based on a circularity of the region, similarity with the airway and proximity to the airway, wherein a region with a highest score is the artery; and computing a mean distance from a center of the artery to boundary points of the artery, wherein the mean distance is half the diameter of the artery.

The tapering index is determined by: plotting the diameter of the airway lumen as a function of voxels along a path from a trachea to a terminal branch of the bronchial tree along which the airway is situated; and computing a slope the diameter of the airway lumen along the path, wherein the tapering index relates to the computed slope.

The first ratio, second ratio and tapering index are scored by: setting a score of the first ratio according to a value of the first ratio; setting a score of the second ratio according to a value of the second ratio; and setting a score of the tapering index according to a value of the tapering index. The method further comprises color-coding the first ratio, second ratio and tapering index according to the value of the first ratio, second ratio and tapering index, respectively.

The foregoing features are of representative embodiments and are presented to assist in understanding the invention. It should be understood that they are not intended to be considered limitations on the invention as defined by the claims, or limitations on equivalents to the claims. Therefore, this summary of features should not be considered dispositive in determining equivalents. Additional features of the invention will become apparent in the following description, from the drawings and from the claims.

### BRIEF DESCRIPTION OF THE DRAWINGS

FIG. 1 is a block diagram illustrating a system for automated airway evaluation according to an exemplary embodiment of the present invention;

FIG. 2 is a flowchart illustrating a method for automated airway evaluation according to an exemplary embodiment of the present invention;

FIG. 3 is a set of images illustrating trachea detection according to an exemplary embodiment of the present invention;

FIG. 4 is a set of images illustrating a gradient computation according to an exemplary embodiment of the present invention;

FIG. 5 is a pair of graphs illustrating a gradient sum of circle parts as a function of circle radius according to an exemplary embodiment of the present invention;

FIG. 6A is a set of images illustrating optimized inner and outer circle radii based on gradient information curves computed in FIG. 5 along an x direction according to an exemplary embodiment of the present invention;

FIG. 6B is a set of images illustrating optimized inner and outer circle radii based on gradient information curves computed in FIG. 5 along a y direction according to an exemplary embodiment of the present invention;

FIG. 7 is a sequence of images illustrating a method for refining a segmentation according to an exemplary embodiment of the present invention;

FIG. 8 is a table illustrating scoring based on broncho-arterial ratios computed according to an exemplary embodiment of the present invention;

FIG. 9 is an image illustrating broncho-arterial ratios for an airway tree of a patient computed according to an exemplary embodiment of the present invention;

FIG. 10 is a pair of graphs illustrating a slope computation according to an exemplary embodiment of the present invention;

FIG. 11 is a table illustrating scoring based on a tapering index computed according to an exemplary embodiment of the present invention;

FIG. 12 is an image illustrating a tapering index for an airway tree of a patient computed according to an exemplary embodiment of the present invention; and

FIG. 13 is a pair of images illustrating interactivity of a system for automated airway evaluation according to an exemplary embodiment of the present invention.

### DETAILED DESCRIPTION OF EXEMPLARY EMBODIMENTS

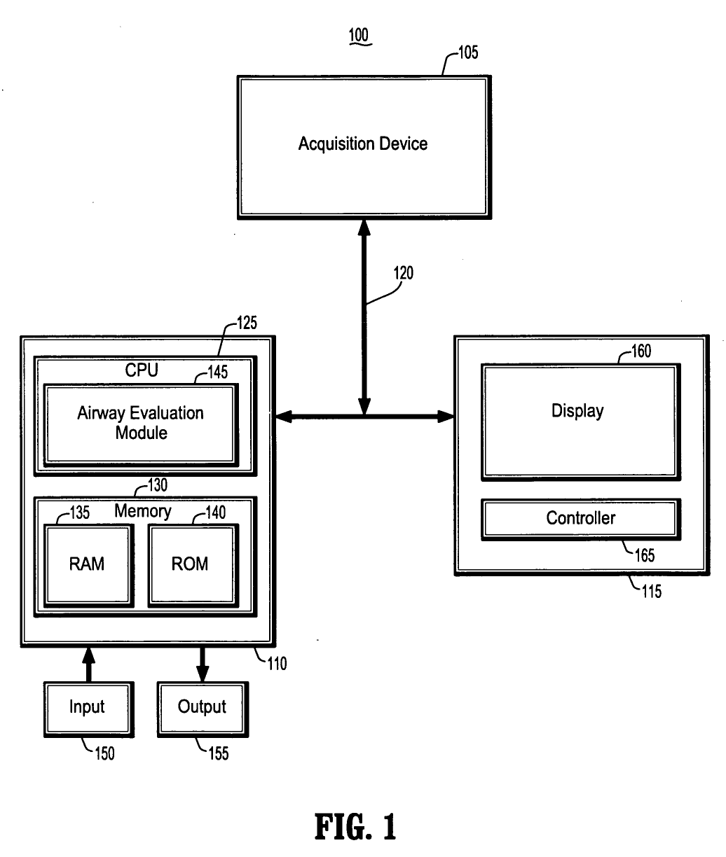

FIG. 1 is a block diagram illustrating a system 100 for automated airway evaluation according to an exemplary embodiment of the present invention. As shown in FIG. 1 the system 100 includes an acquisition device 105, a PC 110 and an operator's console 115 connected over a wired or wireless network 120.

The acquisition device 105 may be a multi-slice computed tomography (MSCT) imaging device or any other three-dimensional (3D) high resolution imaging device such as a magnetic resonance (MR) scanner.

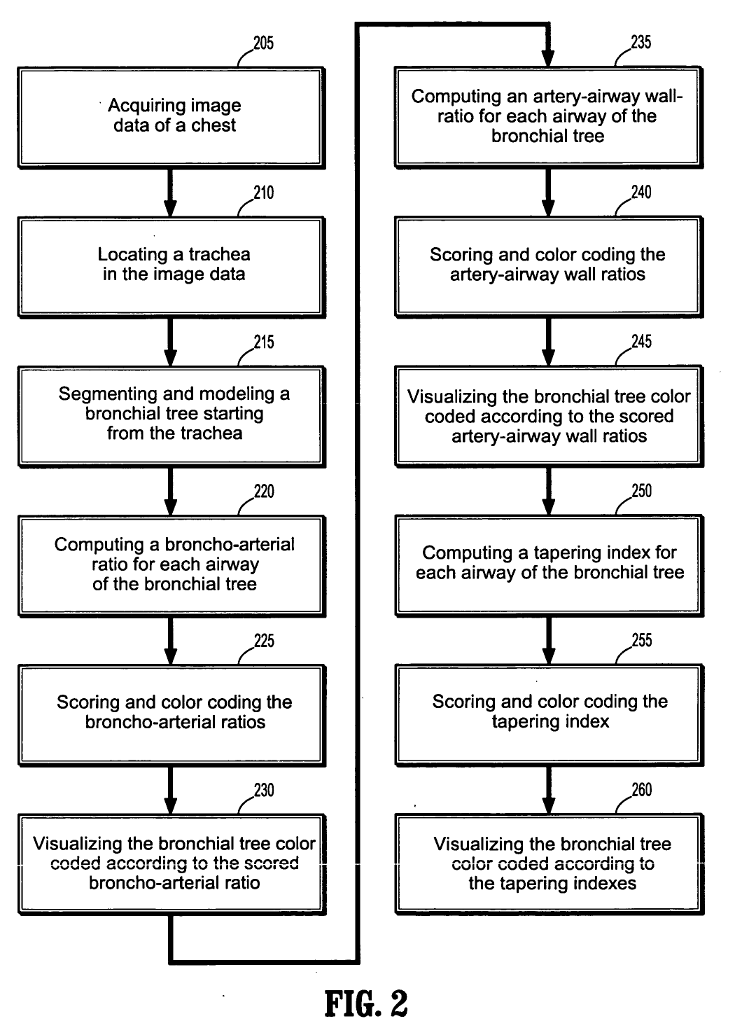

The PC 110, which may be a portable or laptop computer, a medical diagnostic imaging system or a picture archiving communications system (PACS) data management station, includes a CPU 125 and a memory 130 connected to an input device 150 and an output device 155. The CPU 125 includes an airway evaluation module 145 that includes one or more methods for automated airway evaluation to he discussed hereinafter with reference to FIGS. 2-13. Although shown inside the CPU 125, the airway evaluation module 145 can be located outside the CPU 125.

The memory 130 includes a RAM 135 and a ROM 140. The memory 130 can also include a database, disk drive, tape drive, etc., or a combination thereof. The RAM 135 functions as a data memory that stores data used during execution of a program in the CPU 125 and is used as a work area. The ROM 140 functions as a program memory for storing a program executed in the CPU 125. The input 150 is constituted by a keyboard, mouse, etc., and the output 155 is constituted by an LCD, CRT display, printer, etc.

The operation of the system 100 can be controlled from the operator's console 115, which includes a controller 165, e.g., a keyboard, and a display 160. The operator's console 115 communicates with the PC 110 and the acquisition device 105 so that image data collected by the acquisition device 105 can be rendered by the PC 110 and viewed on the display 160. It is to be understood that the PC 110 can be configured to operate and display information provided by the acquisition device 105 absent the operator's console 115, using, e.g., the input 150 and output 155 devices to execute certain tasks performed by the controller 165 and display 160.

The operator's console 115 may further include any suitable image rendering system/tool/application that can process digital image data of an acquired image dataset (or portion thereof) to generate and display images on the display 160. More specifically, the image rendering system may be an application that provides rendering and visualization of medical image data, and which executes on a general purpose or specific computer workstation. It is to be understood that the PC 110 can also include the above-mentioned image rendering system/tool/application.

FIG. 2 is a flowchart showing an operation of a method for automated airway evaluation according to an exemplary embodiment of the present invention. As shown in FIG. 2, 3D image data of a bronchial tree is acquired from a patient (205). This is done, e.g., by using the acquisition device 105, which is operated at the operator's console 115, to scan the patient's chest thereby generating a series of two-dimensional (2D) image slices associated with the chest. The 2D image slices are then combined to form a 3D image of the bronchial tree.

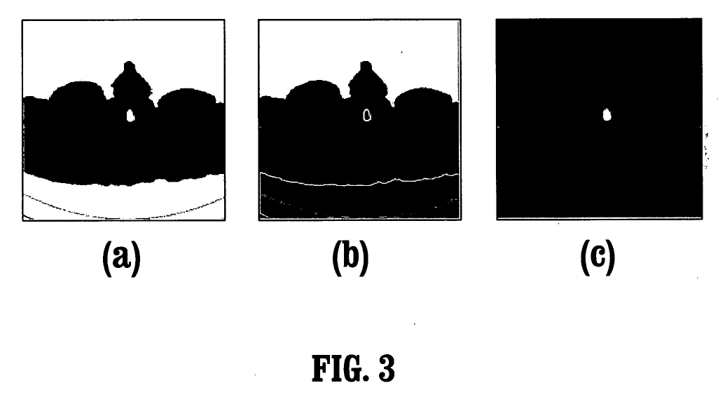

The system 100 then automatically finds a trachea (210). For example, starting on a fifth slice of the image dataset (with the patient oriented head to toe), a threshold is used on air regions below −400 Hounsfield Units (HU) and a smallest region around the center of the image. A region growing process is used on a few slices to confirm that the 2D size of the region does not increase more than 10% from slice to slice. These steps are illustrated in FIG. 3.

In FIG. 3, image (a) illustrates the use of the threshold to select dark (e.g., air-like regions) in the fifth slice, image (b) illustrates a connected-component labeling and image (c) illustrates selection of a limited-size region around the center of the slice (excluding regions close to slice borders). The trachea is illustrated by the white circle in the center of images (a-c). The black regions in images (a-c) illustrate a patient's chest. It is to be understood that the value of the threshold (e.g., −400 HU) is adjustable. Further, voxels above the threshold correspond to solid objects such as blood, soft tissue, etc.

After the trachea has been detected, the bronchial tree is segmented and modeled (215). The bronchial tree can be segmented using any number of suitable segmentation techniques. For example, the bronchial tree can be segmented by using the technique described in Kiraly A. P., McLennan G., Hoffman E. A., Reinhardt J. M., and Higgins W. E., (2002) “Three-dimensional human airway segmentation methods for clinical virtual bronchoscopy” Academic Radiology, 2002. 9(10): p. 1153-1168, a copy of which is incorporated by reference herein in its entirety. It is to be understood that this segmentation is now fully automated by using automatic filtering.

The bronchial tree is modeled based on the algorithm described in Kiraly A. P., Helferty J. P., Hoffman E. A., McLennan G. and Higgins W. E. (2004) “Three dimensional path planning for virtual bronchoscopy” in IEEE Transactions on Medical Imaging. vol. 23, no. 1, November 2004: p. 1365-1379, a copy of which is incorporated by reference herein in its entirety. This method is based on a skeletonization followed by refinement steps to create a smooth tree model of the segmented airway tree. The result is a hierarchical description of the tree as a connected series of branches. Each branch is described by a series of sites. In addition to containing positional information, each site also contains orientation information of the branch at that point.

In the lungs, each airway is accompanied by a corresponding artery. The diameters of healthy airways vary depending on generation number, with the airways decreasing as the airway generation increases. Similarly, the diameter of arteries decreases as the generation increases. In healthy lungs, the diameter of an airway should be roughly equivalent to the diameter of its accompanying artery. If the airway diameter is significantly larger than the artery, this indicates that the patient's airways are probably abnormally dilated. This condition is known as bronchiectasis. An alternative explanation is that the artery is abnormally constricted.

Given the computed tree model, a broncho-arterial ratio is computed for each of the airways (220). This is done, e.g., by selecting every terminal branch of the tree corresponding to the last branch generation of the segmentation. The selected branches are characterized by a number of sites as well as an orientation. For each terminal branch of the tree, a 3D segmentation of the airway as described in U.S. Patent Application entitled, “System and Method for Determining a Size of an Airway Lumen and a Thickness of an Airway Wall”, which claims the benefit of U.S. Provisional Application No. 60/713,025, filed Aug. 31, 2005, is applied, a copy of which is incorporated herein by reference in its entirety. The accompanying artery is detected by using a score as described in Odry B. L., Kirally A. P., Novak C. L., Naidich D. P., Lerallut J-F (2005) “A visualization tool for global evaluation of bronchiectasis and local evaluation of the airways” European Medical & biological Engineering conference EMBEC'05 Proceedings; Prague, November 2005, and then, the artery is segmented as described, e.g., in Kiraly A. P., McLennan G., Hoffman E. A., Reinhardt J. M., and Higgins W. E., (2002) “Three-dimensional human airway segmentation methods for clinical virtual bronchoscopy” Academic Radiology, 2002. 9(10): p. 1153-1168. These processes will now be described in more detail.

Using the tree model, a centerline of a selected airway is selected, a maximum size of a tube is set and a 3D gradient of a volume of the airway within the maximum size of the tube is computed. This is done, e.g., by computing a 3D gradient of the volume within the largest tube (e.g., with the maximum size of the tube), using the gradient formula in Cartesian coordinates:

$$∇ ϕ ( x , y , z ) = \frac{\partial ϕ}{\partial x} {x}^{̑} + \frac{\partial ϕ}{\partial y} {y}^{̑} + \frac{\partial ϕ}{\partial z} {z}^{̑} , \qquad ( 1 )$$

with x, y and z being the principal axes.

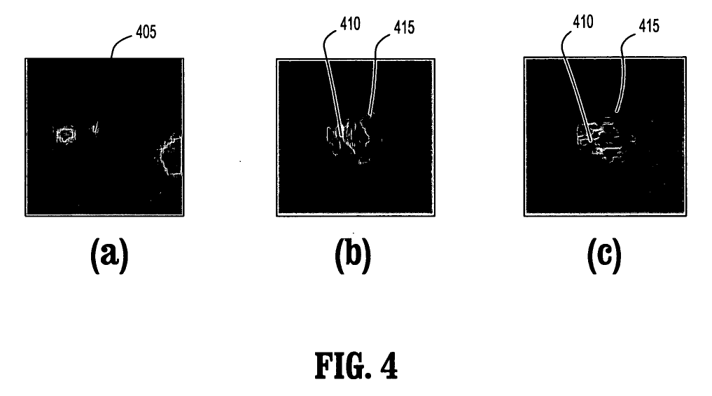

The gradient computation is performed along the axes of the tube. The gradient may be computed using data reformatted with trilinear interpolation. For example, in FIG. 4, by using reformatted data to allow the airway to run perpendicular to the z-axis, inner and outer borders 410 and 415 of an airway 405 (identified by a faint white circle in the center of image (a)) can be viewed as shown in images (b) and (c). In FIG. 4, image (a) illustrates a cross-section of the airway 405 at the center of the image and images (b) and (c) illustrate the cross-section of a 3D gradient computed along the x and y directions within the tube, respectively.

The tube is then positioned along the centerline. It is to be understood that the middle two-thirds of the airway branch is segmented to avoid a bifurcation in the airway. With the tube on the centerline, the tube is grown or expanded by iteratively increasing its radii. For example, starting from radii=zero, the tube is expanded by a quarter of an isotropic voxel. At each iteration, the gradients are checked at the boundary of the tube, thereby enabling inner and outer radii that fit the airway's lumen and wall shapes to be determined.

When the tube is grown, it is separated into four sections. For example, right and left sections for the gradient computed in the x direction and top and bottom sections for the gradient computed in the y direction. The sum of each circle half for each value of the radius is monitored. In addition, the mean gradient value for each of the four sections is computed by using the four mean gradient values at the boundaries of the respective sections at each iteration that increase the radii. It is to be understood that the use of circle halves reduces a partial volumes effect since the curves are computed using the gradients of the x and y directions. This increases the chances of finding a missing or discontinued boundary since a region targeted by the half circles is larger than a region concerned with partial volume effects.

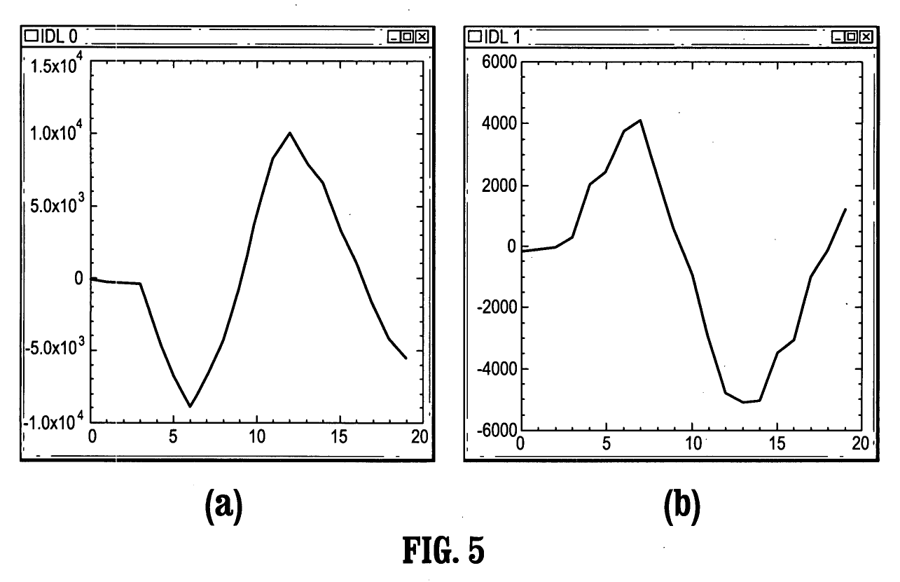

By plotting the gradient sum along the circle halves as a function of radius as shown by the graphs in FIG. 5, a peak analysis of the graphs enables the inner and outer diameters of the tube in the right, left, bottom and top directions to be determined. For example, in FIG. 5, graph (a) illustrates the gradient sum of the circle parts as a function of radius along the x direction and graph (b) illustrates the gradient sum of the circle parts as a function of radius along the y direction. The minimum and maximum peaks of these graphs correspond to the inner and outer radii of the tube, respectively, that best fit the airway.

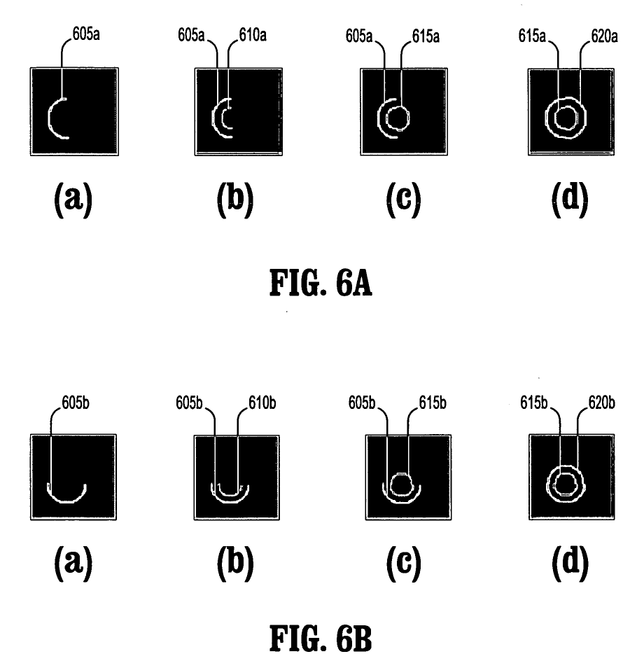

FIGS. 6A and 6B illustrate optimized inner and outer tube diameters found within the gradient information curves along the x and y directions, respectively. In FIGS. 6A and 6B, curves 605a,b of image (a) illustrate a first half outer radius of the tube, curves 610a,b of image (b) illustrate a first half inner diameter of the tube, curves 615a,b of image (c) illustrate a last half inner diameter of the tube thereby completing the full inner diameter and curves 620a,b of image (d) illustrate a last half outer diameter of the tube thereby completing the full outer diameter.

The segmentation is then adjusted by using the selected inner and outer diameters. In other words, the tube is fit to the airway by using the determined inner and outer radii. This is done, e.g., by validating points from the gradients along the x and y directions that are common to the inner and outer diameters as part of the segmentation. In most cases, both boundaries have common points that are confirmed as part of the airway wall or lumen diameter.

For the rest of the points, e.g., for points that are only part of diameters of the gradient along the x direction or the gradient along the y direction but not both, a threshold corresponding to a third of the maximum value of the x and y gradients is computed. For each of the points below the threshold, corresponding gradient values at the same location are checked and if the value is greater than the threshold value, the point is kept otherwise it is discarded.

Since this process is done separately for the inner and outer diameters, it can leave discontinuities or holes along delineations of the inner and outer diameters. To fix this, the discontinuities or holes are filled. This is done, e.g., by taking farthest points from the centerline and filling the holes between the neighboring points.

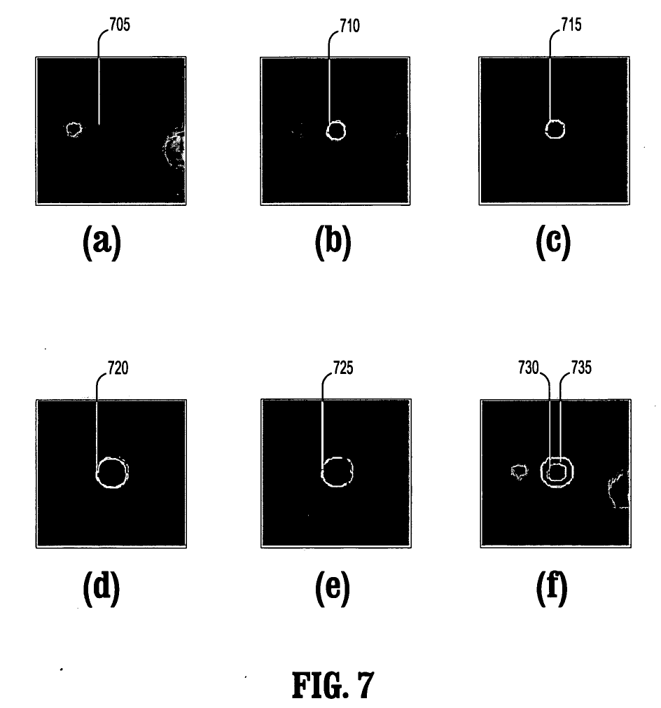

FIG. 7 is a sequence of images illustrating a method for refining a segmentation according to an exemplary embodiment of the present invention. Image (a) illustrates an original cross-section of an airway 705, which is identified by a faint white circle in the center of the image that is to be segmented. Image (b) illustrates a lumen of the airway, wherein the lumen is segmented using the x and y gradients. In this image, inner and outer diameter halves 710 from the x and y gradients are merged. Image (c) illustrates the lumen segmented after refining the gradient. The merged inner diameter halves 715 of image (b) are pruned (e.g., they undergo a thresolding, gradient comparison and verification). Image (d) illustrates the segmentation of a wall of the airway using the x and y gradients. In this image, merged outer diameter circle halves 720 are shown. Image (e) illustrates wall segmentation after pruning. The merged outer diameter circle halves 720 are pruned. Image (f) illustrates the final segmentation of images (b-e) after hole filling. Inner and outer diameters 730 and 735 of the airway are shown in the center of image (f).

Since a tube is used for segmentation, this limits the diameter differences that may be present along the airway. To compensate for this, a tolerance regarding a difference from a diameter map of the airway is set to introduce a smoothness to the deformation of the tube resulting from the gradient process. For the inner tube, the tolerance can be defined as Tolᵢ=5%*diamᵢ with diamᵢ being the diameter found by the diameter map and “i” being the index of a corresponding centerline point. The tolerance could also be expressed by using the highest diameter along the airway diameter map: Tol=5%*max(diamᵢ). The tolerance is positive and the minimum diameter is the minimum from the diameter map. For the outer tube, a median filter on the radii can be used to remove any large radii.

As previously discussed, the diameter of an airway should be roughly equivalent to the diameter of the accompanying artery. The adjacent artery is detected using a score based on the surrounding structures of the airway and the maximum diameter of the artery is calculated and used to compute the broncho-arterial ratio. It is to be understood that scoring is based on orientation similarity between the structure and the airway, circularity of the structure and proximity of the structure to the airway.

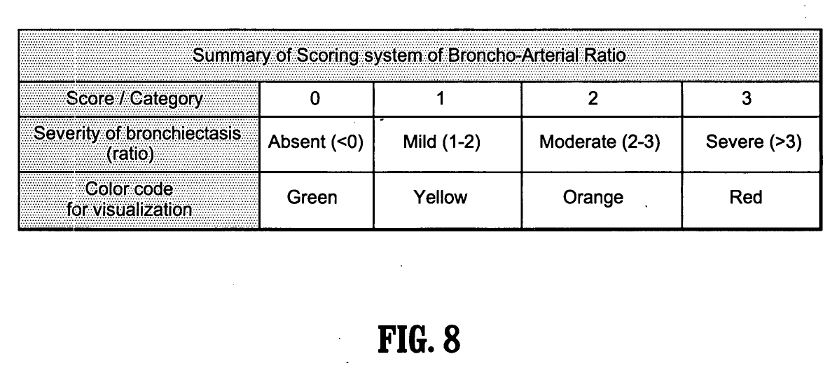

Since the bronco-arterial ratio is an indication of dilation of the airway, it is used to score the severity of the dilation and presence of bronchiectasis as described in Bhalla M., Turcios N., Aponte V., Jenkins M., Leitman B., McCauley D. and Naidich D. (1991) “Cystic Fibrosis: Scoring system with Thin Section CT” Radiology 1991 179:783-788. The score is set depending on the value of the ratio as shown, e.g., by the table in FIG. 8.

In the table, the definition of the categories allows a classification of every terminal branch of the bronchial tree. The scores are then used to sample the whole lung and to color each terminal branch with a corresponding color (225). The bronchial tree is then visualized to identify the location of abnormalities (230).

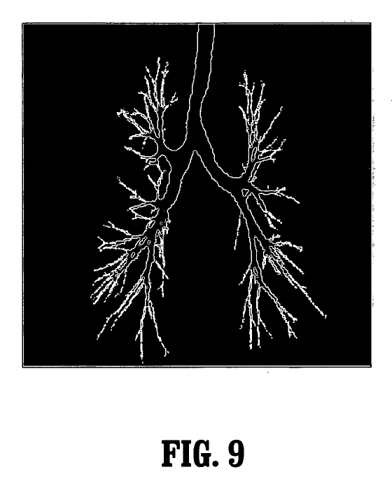

For example, in FIG. 9, the terminal branches of the tree are color coded according to the automatically computed broncho-arterial ratio. In FIG. 9, green (shown as a darker shade of white at branch ends in the black and white image) indicates a normal broncho-arterial ratio of approximately one, yellow (shown as a brighter shade of white at the branch ends in the black and white image) indicates a mildly elevated ratio around two and orange or red (not shown) indicate a moderate to severe ratio greater than two.

Just as the lumen diameter decreases as airway generation increases, the airway wall thickness also decreases with increasing airway generations. Further, just as with broncho-arterial ratio, the thickness of the airway walls should not exceed the diameter of their adjacent artery. When the thickness of an airway wall exceeds the diameter of its adjacent artery, this indicates abnormal airway thickening, which is associated with conditions such as emphysema and chronic obstructive pulmonary disorder (COPD).

After the broncho-arterial ratio is computed for each of the airways, an artery-airway wall-ratio is computed (235). This procedure is very similar to the broncho-arterial ratio computation. For example, the airway wall segmentation is done while segmenting the airway as described above in step 220. The same principals as described in U.S. Provisional Application No. 60/717,669, entitled “Automatic Airway and Artery Grouping Method for Quantitative Analysis”, filed Sep. 16, 2005, a copy of which is incorporated by reference herein in its entirety, are applied to detect the artery and compute the diameter as will now be described.

For example, in a 2D cross-section around the central point of the airway centerline, the algorithms selects all high-density regions (e.g., blood, etc. but not air) close to the airway. It is to be understood that the size of the cross-section varies with the estimated diameter of the selected airway. For each of the regions, features are computed that are specific to the adjacent artery. These features may include circularity (since the cross section of the artery is assumed to be a circle), proximity to the airway and 3D orientation similarity (to confirm that the artery is parallel to the airway). The sum of these features allows for the selection of the artery that should have the best score for all the regions. The artery diameter is computed by taking the mean distance from the center of the artery to its boundaries.

Scoring is done as described above with reference to the table of FIG. 8 (240), and in the visualization of the bronchial tree, the terminal branch is colored according to the codes shown in the table (245).

In a person with healthy airways, the airway lumen decreases or tapers as the airway generation increases. In patients with abnormally dilated lumens, the lumen diameter will remain constant or even increase as the airway generation increases. Bronchiectasis is characterized by the lack of tapering anywhere along the airway path.

Now that the artery-airway wall ratio is computed, a tapering index is computed 4(250). This is done, e.g., by extracting from the tree model, every tree path from the trachea to each terminal branch to obtain all sites or voxels along each path. From the bronchial tree segmentation, a diameter map that represents the highest diameter values along the tree is computed as described in Kiraly A. P., Helferty J. P., Hoffman E. A., McLennan G. and Higgins W. E. (2004) “Three dimensional path planning for virtual bronchoscopy” in IEEE Transactions on Medical Imaging. vol. 23, no. 1, November 2004: p. 1365-1379. This map colors the voxels in the lumen according to the maximum diameter at that location in the airway. Thus, for each site of the path, there is a corresponding lumen diameter. It is to be understood that the lumen diameter for site can be computed as well.

For each path, there is now a graph of diameter as a function of the sites. To estimate the tapering index, multiple slopes of a diameter curve along the path are calculated. The diameter curve is divided into four segments (e.g., right and left sections for the gradient computed in the x direction and top and bottom sections for the gradient computed in the y direction) to compute the slope. A global approach can be chosen here by emphasizing the whole path and not a specific branch. However, specific branches can be selected from along the path and a slope for each generation can be computed. This allows an intra-comparison of multiple branches from the same generation that is also an indicator of a lack of tapering.

To compute the slope, a linear fitting function is applied to the curve segments (data xᵢ, yᵢ) on the model y=Ax+B. The linear fitting function minimizes the chi-square error statistic:

$${χ}_{Obs}^{2} = {\sum }_{cells} \frac{{( O - E )}^{2}}{E}$$

with O representing the observed frequency cell and E the expected frequency cell of the contingency table. Frequency tables of two variables presented simultaneously are called contingency tables. Contingency tables are constructed by listing all the levels of one variable as rows in a table and the levels of the other variable as columns, then finding the joint or cell frequency for each cell.

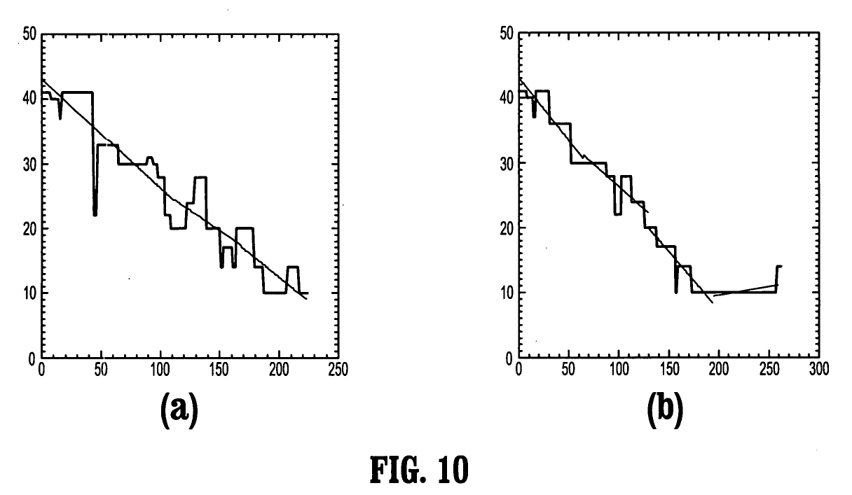

An example of the slope computation is shown in FIG. 10. In this example, diameter is in tens of mm as a function of the number of sites. Graph (a) illustrates a regular tapering along a path. Graph (b) illustrates a lack of tapering at the end of the path. It is to be understood that a path that tapers should have a significant negative slope (e.g., less than −0.15). A path that has a slope near zero indicates that the lumen diameter is remaining roughly constant with increasing airway generation, a condition known as cylindrical bronchiectasis. Slopes that are significantly positive (e.g., greater than 0.15) mean that the lumen diameter is actually increasing with increasing airway generation, indicating moderate to severe bronchiectasis.

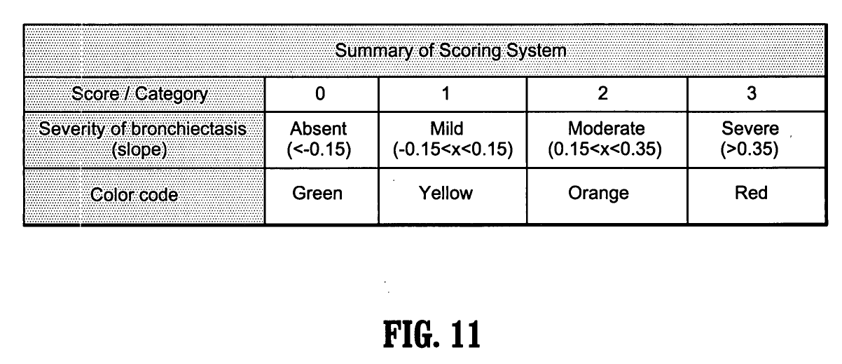

A score is defined depending on the highest slope along the path as shown by the table of FIG. 11 and a color-coding based on the score is defined (255). This score or tapering index is used to visualize a lack of tapering along the paths of the bronchial tree by coloring each terminal branch based on the score for the whole path (260).

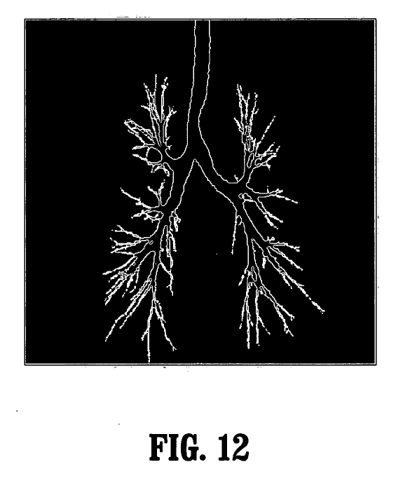

An example of this visualization is shown in FIG. 12. In FIG. 12, green (shown as a darker shade of white at branch ends in the black and white image) indicates that the airway path leading to this branch shows normal tapering of the airway diameters, yellow (shown as a brighter shade of white at the branch ends in the black and white image) indicates that the path leading to this branch shows a mild lack of tapering and orange or red (not shown) indicate a severe lack of tapering.

By applying the color coding as described above with reference to FIGS. 9 and 12, one can visually detect where problems occur within the bronchial tree and their severity. In addition, the color coding can help determine any patterns of the disease affecting a specific region of the lung or a specific lobe. Further, the color coding can indicate how widespread the abnormalities are by indicating whether a few or many abnormalities are affected by bronchiectasis.

It is to be understood that the color-coded trees can be interactively displayed on the display 160, thereby allowing a user to rotate the tree and zoom in or out to inspect the tree from all sides. In addition, the user may click on the tree at any point, and another window will display the corresponding point in the original CT data as well as a local zoomed view of a 3D rendering of the selected airway and the adjacent artery and scores for that artery. The user can also click on an airway in the original CT data and the corresponding location can be indicated on the 3D tree display.

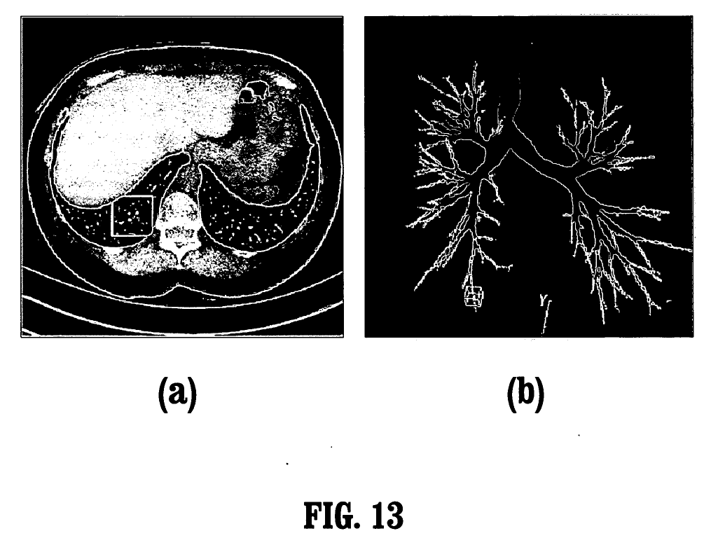

For example, as shown in image (b)) of FIG. 13, is user can click on a specific airway highlighted by the white box. A corresponding lung axial slice is displayed and the airway is highlighted by a rectangle as show in image (a). In addition, during the visualization, a user can toggle between the depictions of the artery-lumen ratios, the artery-wall ratios or the tapering index.

A system and method for automated airway evaluation according to an exemplary embodiment of the present invention enables a global assessment of the airways to provide quantitative assessment of the decree of airway tapering and associated broncho-arterial and broncho wall-arterial ratios. For example, the system is able to detect the presence and location of mild bronchiectasis characterized by lack of lumen tapering and elevated broncho-arterial ratios. The quantitative measurements are then visualized in an interactive 3D model of the airway tree graphically displaying the location and extent of disease. For example, the 3D visualizations of the quantitative measurements of airway tapering and broncho-arterial ratio show a good correlation between these complementary measurements of abnormal airway dilation. In addition, employing a 3D airway segmentation technique by using tube fitting along the x and y gradients allows direct computation of both inner and outer airway diameters, thereby reducing sensitivity to partial volume artifacts.

It is to be further understood that the present invention may be implemented in various forms of hardware, software, firmware, special purpose processors, or a combination thereof. In one embodiment, the present invention may be implemented in software as an application program tangibly embodied on a program storage device (e.g., magnetic floppy disk, RAM, CD ROM, DVD, ROM, and flash memory). The application program may be uploaded to, and executed by, a machine comprising any suitable architecture.

It is to be further understood that because some of the constituent system components and method steps depicted in the accompanying figures may he implemented in software, the actual connections between the system components (or the process steps) may differ depending on the manner in which the present invention is programmed. Given the teachings of the present invention provided herein, one of ordinary skill in the art will be able to contemplate these and similar implementations or configurations of the present invention.

It should also be understood that the above description is only representative of illustrative embodiments. For the convenience of the reader, the above description has focused on a representative sample of possible embodiments, a sample that is illustrative of the principles of the invention. The description has not attempted to exhaustively enumerate all possible variations. That alternative embodiments may not have been presented for a specific portion of the invention, or that further undescribed alternatives may be available for a portion, is not to be considered a disclaimer of those alternate embodiments. Other applications and embodiments can be implemented without departing from the spirit and scope of the present invention.

It is therefore intended, that the invention not be limited to the specifically described embodiments, because numerous permutations and combinations of the above and implementations involving non-inventive substitutions for the above can be created, but the invention is to be defined in accordance with the claims that follow. It can be appreciated that many of those undescribed embodiments are within the literal scope of the following claims, and that others are equivalent.

What is claimed is:

1. A method for evaluating an airway in a bronchial tree, comprising:
segmenting a bronchial tree;
modeling the segmented bronchial tree;
computing a first ratio for an airway in the segmented and modeled bronchial tree, wherein the first ratio is a ratio between a diameter of the airway lumen and a diameter of an artery accompanying the airway; computing a second ratio for the airway, wherein the second ratio is a ratio between the diameter of the artery and a thickness of the airway wall; or computing a tapering index for the airway, wherein the tapering index indicates a tapering of the diameter of the airway lumen;
scoring and color coding the first ratio, second ratio or tapering index; and
visualizing the segmented and modeled bronchial tree color coded according to the first ratio, second ratio or tapering index.

2. The method of claim 1, wherein segmenting the bronchial tree comprises:
applying a filtered adaptive threshold region growing to the bronchial tree starting from a seed point in a trachea.

3. The method of claim 1, wherein modeling the segmented bronchial tree comprises:
defining a skeleton of the segmented bronchial tree;
performing a multistage refinement of the skeleton to arrive at a tree structure; and
computing a diameter map of the tree structure.

4. The method of claim 1, wherein the diameter of the airway lumen and the thickness of the airway wall are determined by:
computing a centerline of the airway;
computing a three-dimensional (3D) gradient of a volume of the airway within a first threshold;
positioning a tube along the centerline;
iteratively expanding the tube by increasing its radius until the radius of the tube reaches the first threshold;
determining inner and outer radii of the tube by checking the 3D gradient computed along an x-axis and a y-axis of the tube at a boundary of the tube at each iteration; and
fitting the tube to the airway by using the determined inner and outer radii, wherein the inner radius of the fit tube is half the diameter of the airway lumen and the outer radius of the fit tube minus the inner radius of the fit tube is the thickness of the airway wall.

5. The method of claim 1, wherein the artery is identified and the diameter of the artery are determined by:
labeling regions of high intensity in a cross-sectional plane of the bronchial tree;
computing a score based on a circularity of the region, similarity with the airway and proximity to the airway, wherein a region with a highest score is the artery; and
computing a mean distance from a center of the artery to boundary points of the artery, wherein the mean distance is half the diameter of the artery.

6. The method of claim 1, wherein the tapering index is determined by:
plotting the diameter of the airway lumen as a function of voxels along a path from a trachea to a terminal branch of the bronchial tree along which the airway is situated; and
computing a slope the diameter of the airway lumen along the path, wherein the tapering index relates to the computed slope.

7. The method of claim 1, wherein the first ratio, second ratio and tapering index are scored by:
setting a score of the first ratio according to a value of the first ratio;
setting a score of the second ratio according to a value of the second ratio; and
setting a score of the tapering index according to a value of the tapering index.

8. The method of claim 8, further comprising:
color-coding the first ratio, second ratio and tapering index according to the value of the first ratio, second ratio and tapering index, respectively.

9. The method of claim 1, further comprising:
acquiring an image of a chest including the bronchial tree by using computed tomography or magnetic resonance imaging.

10. A system for evaluating an airway in a bronchial tree, comprising:
a memory device for storing a program;
a processor in communication with the memory device, the processor operative with the program to:
segment a bronchial tree;
model the segmented bronchial tree;
compute a first ratio for an airway in the segmented and modeled bronchial tree, wherein the first ratio is a ratio between a diameter of the airway lumen and a diameter of an artery accompanying the airway; compute a second ratio for the airway, wherein the second ratio is a ratio between the diameter of the artery and a thickness of the airway wall; or compute a tapering index for the airway, wherein the tapering index indicates a tapering of the diameter of the airway lumen;
score and color code the first ratio, second ratio or tapering index; and
visualize the segmented and modeled bronchial tree color coded according to the first ratio, second ratio or tapering index.

11. The system of claim 10, wherein when segmenting the bronchial tree the processor is further operative with the program to:
apply a filtered adaptive threshold region growing to the bronchial tree starting from a seed point in a trachea.

12. The system of claim 10, wherein when modeling the segmented bronchial tree the processor is further operative with the program to:
define a skeleton of the segmented bronchial tree;
perform a multistage refinement of the skeleton to arrive at a tree structure; and
compute a diameter map of the tree structure.

13. The system of claim 10, wherein when determining the diameter of the airway lumen and the thickness of the airway wall the processor is further operative with the program to:
compute a centerline of the airway;
compute a three-dimensional (3D) gradient of a volume of the airway within a first threshold;
position a tube along the centerline;
iteratively expand the tube by increasing its radius until the radius of the tube reaches the first threshold;
determine inner and outer radii of the tube by checking the 3D gradient computed along an x-axis and a y-axis of the tube at a boundary of the tube at each iteration; and
fit the tube to the airway by using the determined inner and outer radii, wherein the inner radius of the fit tube is half the diameter of the airway lumen and the outer radius of the fit tube minus the inner radius of the fit tube is the thickness of the airway wall.

14. The system of claim 10, wherein when the artery is identified and the diameter of the artery are determined the processor is further operative with the program to:
label regions of high intensity in a cross-sectional plane of the bronchial tree;
compute a score based on a circularity of the region, similarity with the airway and proximity to the airway, wherein a region with a highest score is the artery; and
compute a mean distance from a center of the artery to boundary points of the artery, wherein the mean distance is half the diameter of the artery.

15. The system of claim 10, wherein when determining the tapering index the processor is further operative with the program to:
plot the diameter of the airway lumen as a function of voxels along a path from a trachea to a terminal branch of the bronchial tree along which the airway is situated; and
compute a slope the diameter of the airway lumen along the path, wherein the tapering index relates to the computed slope.

16. The system of claim 10, wherein when scoring the first ratio, second ratio and tapering index the processor is further operative with the program to:
set a score of the first ratio according to a value of the first ratio;
set a score of the second ratio according to a value of the second ratio; and
set a score of the tapering index according to a value of the tapering index.

17. The system of claim 16, wherein the processor is further operative with the program code to:
color-code the first ratio, second ratio and tapering index according to the value of the first ratio, second ratio and tapering index, respectively.

18. The system of claim 10, wherein the processor is further operative with the program code to:
acquire an image of a chest including the bronchial tree by using a computed tomography or magnetic resonance imaging device.

19. A method for automatically evaluating multi-slice computed tomography (MSCT) image data of a bronchial tree, comprising:
segmenting and modeling the bronchial tree starting from a trachea;
computing a first ratio for each airway of the bronchial tree, wherein the first ratio is a ratio between a diameter of the airway lumen and a diameter of an artery accompanying the airway;
scoring and color coding the first ratio;
visualizing the segmented and modeled bronchial tree color coded according to the first ratio;
computing a second ratio for each airway of the bronchial tree, wherein the second ratio is a ratio between the diameter of the artery and a thickness of the airway wall;
scoring and color coding the second ratio;
visualizing the segmented and modeled bronchial tree color coded according to the second ratio;
computing a tapering index for each airway of the bronchial tree, wherein the tapering index indicates a tapering of the diameter of the airway lumen;
scoring and color coding the tapering index; and
visualizing the segmented and modeled bronchial tree color coded according to the tapering index.

20. The method of claim 19, wherein the diameter of the airway lumen and the thickness of the airway wall are determined by:
computing a centerline of the airway;
computing a three-dimensional (3D) gradient of a volume of the airway within a first threshold;
positioning a tube along the centerline;
iteratively expanding the tube by increasing its radius until the radius of the tube reaches the first threshold;
determining inner and outer radii of the tube by checking the 3D gradient computed along an x-axis and a y-axis of the tube at a boundary of the tube at each iteration; and
fitting the tube to the airway by using the determined inner and outer radii, wherein the inner radius of the fit tube is half the diameter of the airway lumen and the outer radius of the fit tube minus the inner radius of the fit tube is the thickness of the airway wall.

21. The method of claim 19, wherein the artery is identified and the diameter of the artery are determined by:
labeling regions of high intensity in a cross-sectional plane of the bronchial tree;
computing a score based on a circularity of the region, similarity with the airway and proximity to the airway, wherein a region with a highest score is the artery; and
computing a mean distance from a center of the artery to boundary points of the artery, wherein the mean distance is half the diameter of the artery.

22. The method of claim 19, wherein the tapering index is determined by:
plotting the diameter of the airway lumen as a function of voxels along a path from a trachea to a terminal branch of the bronchial tree along which the airway is situated; and
computing a slope the diameter of the airway lumen along the path, wherein the tapering index relates to the computed slope.

23. The method of claim 19, wherein the first ratio, second ratio and tapering index are scored by:
setting a score of the first ratio according to a value of the first ratio;
setting a score of the second ratio according to a value of the second ratio; and
setting a score of the tapering index according to a value of the tapering index.

24. The method of claim 23, further comprising:
color-coding the first ratio, second ratio and tapering index according to the value of the first ratio, second ratio and tapering index, respectively.
